(19) United States
(12) Patent Application Publication
(10) Pub. No.: US 2007/0169216 A1
(43) Pub. Date: Jul. 19, 2007

(54) Novel Cucurbita plants

(75) Inventors: Jean Louis Marie Edouard Nicolet, Sarrians (FR); Kevin Cook, Naples, FL (US); Gabriele Gusmini, Naples, FL (US)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(73) Assignee: Syngenta Participations AG

(21) Appl. No.: 11/405,975

(22) Filed: Apr. 18, 2006

Related U.S. Application Data
(60) Provisional application No. 60/758,816, filed on Jan. 13, 2006.

(51) Int. Cl. A01H 5/00; C12N 15/82

(52) U.S. Cl. 800/278; 800/310

(57) ABSTRACT

The present invention relates to novel Cucurbita plants comprising a new gene conferring resistance to viruses upon said plant, in particular resistance to Cucumber Mosaic Virus (CMV). The new gene is designated as Cmv-2. The present invention also relates to seeds and parts of said plants, for example fruits. The present invention also relates to methods of making and using such plants.

### CROSS-REFERENCE TO RELATED APPLICATIONS

This application claims the benefit of U.S. Provisional Application No. 60/758,816, filed Jan. 13, 2006. The above application is incorporated herein by reference in its entirety.

### FIELD OF THE INVENTION

The present invention relates to novel Cucurbita plants resistant to viruses. The present invention also relates to seeds and parts of said plants, for example fruits. The present invention also relates to methods of making and using such plants.

### BACKGROUND OF THE INVENTION

The Cucurbita are members of the family Cucurbitaceae, which comprises about 90 genera and 700 to 760 species, mostly of the tropics. The genus Cucurbita includes four major species, pepo, argyrosperma, moschata, and maxima, one minor species, ficifolia, and some wild ones. The major commercial crops of the genus Cucurbita are squash and pumpkin.

Squash and pumpkins are unique in that they represent several species for the same crop. They encompass many summer and winter edible types as well as ornamental gourds. Summer squash is Cucurbita pepo, but winter squash may be C. pepo (e.g. ‘Acorn’), C. moschata (‘Butternut’), C. mixta (‘Japanese Pie’), or C. maxima (‘Hubbard’, etc.). The Jack‘O’ Lantern type of pumpkin is C. pepo, but commercially canned pumpkin pie mix is also made from C. moschata (e.g. ‘Libby Select’) or C. maxima (‘NK 580’). C. mixta and C. ficifolia are used for food in Mexico and in Central and South American countries.

Squash and pumpkin are usually grown for their fruit, harvested immature for summer squash or mature for winter squash and pumpkin. Summer squash is most often eaten fresh, but winter squash may be stored several months. Both summer and winter squash are canned and frozen commercially. It has been reported that the world production of Cucurbita in 1979-81 was 5,256,000 metric tons from 539,000 acres annually.

Several types of viruses affect crops of Cucurbita plants, for example Zucchini Yellow Mosaic Virus (ZYMV), Watermelon Mosaic Virus (WMV), and Papaya Ringspot Virus (PRSV). Cucumber Mosaic Virus (CMV), which belongs to the genus of the Cucumoviruses, family Bromoviridae (Roossinck (2002) Journal of Virology 76: 3382-3387), also affects crops of Cucurbita plants.

These viruses cause extensive damages to Cucurbita crops and new strategies to control them are always desired. There is therefore an unfulfilled need for new sources of genetic resistance to viruses in Cucurbita, in particular new sources of resistance to CMV in Cucurbita.

### SUMMARY OF THE INVENTION

The instant invention addresses the unfulfilled need for new sources of resistance to viruses in Cucurbita, in particular the unfulfilled need for new sources of resistance to CMV in cucurbita. To address these needs, the instant invention provides novel Cucurbita plants comprising a new gene conferring resistance to viruses upon said plants, in particular resistance to CMV.

The new gene, designated Cmv-2, was surprisingly found in C. moschata var. Seminole pumpkin and successfully transferred to other Cucurbita plants using embyo rescue and backcross techniques. The Cmv-2 gene is located at the chromosomal locus of the gene conferring resistance to CMV in C. moschata var. Seminole pumpkin or in inbred line 90-3588, representative seed of which being deposited under accession number NCIMB 41369. Accordingly, a plant according to the present invention is a plant comprising a Cmv-2 gene. The Cmv-2 gene confers upon a Cucurbita plant high level of virus resistance. The resistance conferred by the Cmv-2 gene is stable and can be transferred to other Cucurbita plants, for example Cucurbita plants susceptible to the virus. In one embodiment, the Cmv-2 gene is obtainable from a plant of C. moschata var. Seminole pumpkin. In one embodiment, the Cmv-2 gene is obtainable from a plant of line 90-3588. In one embodiment, the Cmv-2 gene is dominant. In one embodiment, resistance to CMV according to the instant invention is monogenic.

In one embodiment, a plant of the invention is a Cucurbita plant comprising a Cmv-2 gene, wherein said Cucurbita plant is not a plant of C. moschata var. Seminole pumpkin. In one embodiment, a plant of the invention is not a C. moschata plant. In one embodiment, a plant of the invention is a plant of C. pepo, a plant of C. maxima or a plant of C. mixta. In one embodiment, a plant of the invention is a squash plant. In one embodiment, a plant of the invention is a summer squash plant, a winter squash plant or a pumpkin plant. In one embodiment, a plant of the invention is a plant of C. pepo L. In one embodiment, a plant of the instant invention is homozygous for a Cmv-2 gene.

The instant invention also provides seeds and parts of the plants of the invention, for example fruits, and methods of making and using such plants. The Cmv-2 gene of the instant invention can also be combined with other resistance strategies to further increases resistance to the virus, for example horizontal resistance.

The instant invention allows growers to prevent or reduce damages to their crops caused by the virus, and also allows for increased flexibility in their resistance strategies. Higher yields are obtained and fruit can be harvested over a longer period of time.

The present invention therefore provides:

A Cucurbita plant comprising a Cmv-2 gene, wherein when said Cucurbita plant is crossed with a plant of inbred line 90-3588 100% of F1 plants resulting from said cross are resistant to CMV, and:
a) 100% of said F1 plants produce 100% of F2 progeny plants resistant to CMV, when said Cucurbita plant is homozygous for a Cmv-2 gene; or
b) 50% of said F1 plants produce 100% of F2 progeny plant resistant to CMV and 50% said F1 plants produce a 3:1 ratio of F2 progeny plants resistant to CMV to F2 progeny plants susceptible to CMV, when said Cucurbita plant is heterozygous for a Cmv-2 gene,

wherein said plant is not a plant of C. moschata cv. Seminole Pumpkin.

In one embodiment, the plant is not a C. moschata plant. In one embodiment, the plant is a C. pepo plant, a C. maxima plant or a C. mixta plant, in one embodiment, a C. pepo L. plant. In one embodiment, the Cmv-2 gene is obtainable from a plant of C. moschata cv. Seminole Pumpkin or from a plant of line 90-3588, representative seed of which being deposited under accession number NCIMB 41369. In one embodiment, the plant is homozygous for said Cmv-2 gene. In one embodiment, the plant is an inbred line, a hybrid or a dihaploid. In one embodiment, the plant is male-sterile.

The present invention further provides:

A C. pepo plant highly resistant to CMV, wherein the plant comprises a Cmv-2 gene. In one embodiment, the plant is a C. pepo L. plant. In one embodiment, the Cmv-2 gene is obtainable from a plant of C. moschata cv. Seminole Pumpkin or from a plant of line 90-3588, representative seed of which being deposited under accession number NCIMB 41369. In one embodiment, the plant is homozygous for said Cmv-2 gene. In one embodiment, the plant is an inbred line, a hybrid or a dihaploid. In one embodiment, the plant is male-sterile. In one embodiment, the plant is identified and defined in an allelic test as described herein.

The present invention further provides:

A Cucurbita plant of inbred line 90-3588, representative seed of which been deposited under accession NCIMB 41369.

The present invention further provides:

Seed or part of anyone of the Cucurbita plants above. In one embodiment, the part is a cell, pollen, ovule or fruit.

The present invention further provides:

A method of producing a fruit of a Cucurbita plant comprising: growing anyone of the Cucurbita plants above until a fruit is produced; and harvesting said fruit.

The present invention further provides:

A method of producing seed of a Cucurbita plant comprising: growing anyone of the Cucurbita plants above; allowing said Cucurbita plant to self-pollinate; and harvesting seeds from said Cucurbita plant.

The present invention further provides:

A method of vegetatively propagating a Cucurbita plant comprising: collecting a tissue from anyone of the Cucurbita plants above; cultivating said tissue to obtain proliferated shoots; rooting said proliferated shoots to obtain rooted plantlets.

The present invention further provides:

A method for producing a Cucurbita plant comprising a Cmv-2 gene comprising: crossing anyone of the Cucurbita plants above and a Cucurbita plant lacking a Cmv-2 gene; obtaining a progeny plant from the cross; and selecting in the progeny a Cucurbita plant comprising a Cmv-2 gene.

The present invention further provides:

A Cucurbita plant comprising a Cmv-2 gene obtainable by the method above.

The present invention further provides:

A method of transferring a Cmv-2 gene to a Cucurbita plant lacking said gene comprising: crossing anyone of the Cucurbita plants above and a Cucurbita plant lacking a Cmv-2 gene; obtaining a progeny plant from the cross; and selecting in said progeny a Cucurbita plant comprising a Cmv-2 gene.

The present invention further provides:

A Cucurbita plant comprising a Cmv-2 gene obtainable by the method above.

The present invention further provides:

A method of conferring resistance to CMV upon a Cucurbita plant comprising: crossing anyone of the Cucurbita plants above and a Cucurbita plant susceptible to CMV; obtaining a progeny plant from the cross; and selecting in said progeny a Cucurbita plant resistant to CMV.

The present invention further provides:

A Cucurbita plant resistant to CMV obtainable by the method above.

The present invention further provides:

A method for producing hybrid seed comprising: crossing a any one of plants above, wherein said plant is an inbred line, with a plant of another inbred line; and harvesting hybrid seed resulting from the cross.

The present invention further provides:

Hybrid seed produced by the method above, wherein said hybrid seed comprises a Cmv-2 gene.

The present invention further provides:

A method for producing a Cucurbita plant comprising a Cmv-2 gene comprising: obtaining a plant of C. moschata cv. Seminole Pumpkin; crossing said plant of C. moschata cv. Seminole Pumpkin plant with a Cucurbita plant lacking a Cmv-2 gene; rescuing an embryo resulting from the cross; regenerating a plant from said embryo; and selecting a plant that is resistant to CMV;

The present invention further provides:

A Cucurbita plant obtainable by the method above, wherein said plant comprises a Cmv-2 gene.

### Definitions

Gene: Unit of inheritance. Genes are located at fixed loci in chromosomes. A gene can exist in a series of alternative forms called alleles. A gene can control or contribute to a trait, for example resistance to a disease.

Trait: characteristic or phenotype, for example resistance to a disease. A trait may be inherited in a dominant or recessive manner. A trait may be monogenic or polygenic, or may also result from the interaction of one or more genes, in some cases with the environment. A trait is for example resistance to one or more viruses, for example CMV.

Cmv-2 gene: gene conferring resistance to CMV upon a Cucurbita plant and located at the chromosomal locus of the gene conferring resistance to CMV in inbred line 90-3588 or in C. moschata var. Seminole pumpkin.

“Tester” plant: plant used to characterize genetically a gene or a trait in a plant to be tested. Typically, the plant to be tested is crossed with a “tester” plant and the segregation ratio of the trait in progeny plants of the cross is scored.

Allele: One of a pair or series of forms of a gene, which are alternative in inheritance because they are situated at the same locus in homologous chromosomes.

Locus: region on a chromosome, which generally comprises a gene, e.g. a gene controlling or contributing to a trait.

Resistance: biological mechanism based on the genotype of a plant, by which a plant exhibits no symptoms, insignificant symptoms, or reduced symptoms of a disease in a resistant plant, as compared to the plants without the resistance (susceptible plants), which are affected by the same disease at the same time, e.g. during the same production cycle, and in the same production unit. Resistance is thus preferably the ability of a plant to reduce the development of a pathogen. (see for example Robinson, R. A., 1969: Review Applied Mycology 48, 593-606).

Resistance to CMV: defined as the ability of a plant to reduce the development of CMV epidemics, whereas susceptible plants are affected by CMV at the same time, e.g. during the same production cycle, and in the same production unit.

Monogenic: determined by a single locus.

Polygenic: determined by more than one locus.

Dominant: results in a complete phenotypic manifestation at heterozygous or homozygous state. However, in some cases a gene dosage effect may be observed, which may result in a slightly stronger and more reproducible phenotype at the homozygous state than at the heterozygous state.

Recessive: manifests itself only when present at homozygous state.

Genetic linkage: association of characters in inheritance due to location of genes in proximity on the same chromosome. Measured by percent recombination (i.e. recombination frequency) between loci (centi-Morgan, cM).

Isogenic: plants, which are genetically identical, except that they may differ by the presence or absence of a gene at a locus conferring a trait or heterologous DNA sequence.

Marker assisted selection: refers to the process of selecting a desired trait or desired traits in a plant or plants by detecting one or more nucleic acids from the plant, where the nucleic acid is associated with the desired trait.

Dihaploid: plants generated by doubling of haploid plants (single chromosome set) (e.g. through anther culture or microspore culture) giving a complete homozygous plant.

As used herein, the term “plant” includes plant cells, plant protoplasts, plant cells of tissue culture from which Cucurbita plants can be regenerated, plant calli, plant clumps, and plant cells that are intact in plants or parts of plants, such as pollen, flowers, seeds, fruits, leaves and the like.

Regeneration: refers to the development of a plant from tissue culture.

Single Gene Converted (Conversion): refers to plants which are developed by a plant breeding technique called backcrossing wherein essentially all of the desired morphological and physiological characteristics of a variety are recovered in the offspring in addition to the single gene transferred into the variety via the backcrossing technique or via genetic engineering.

### DETAILED DESCRIPTION OF THE INVENTION

The instant invention addresses the unfulfilled need for new sources of resistance to viruses in Cucurbita, in particular the unfulfilled need for new sources of resistance to CMV in Cucurbita. To address these needs, the instant invention provides novel Cucurbita plants comprising a new gene conferring resistance to viruses upon said plants. In particular, the new gene of the instant invention confers resistance to CMV upon said Cucurbita plants. The instant invention also provides seeds and parts of the plants of the invention, for example fruits. The present invention also provides methods of making and using such plants.

The plants of the present invention comprise a gene conferring upon said plants resistance to CMV and designated as Cmv-2. The Cmv-2 gene was unexpectedly found in C. moschata var. Seminole pumpkin (also referred herein to as C. moschata cv. Seminole Pumpkin) and transferred to other Cucurbita plants.

Seminole Pumpkin is a winter squash, species C. moschata. This squash is originally from the Southeastern United States, and its original name, Chassa howitska, means “hanging pumpkin”. Seminole Pumpkin plants can be grown as climbing vines over trees, with the pumkin fruit hanging down from the branches, out of reach of livestock and rot. The leaves of this winter squash are rounded, slightly lobed, medium to dark green in color, with a grayish mottling on the older leaves. The vines are large and bear bell-shaped, cream fruits, approximately 10 to 12 cm wide at the bulb. Generally, fruits are similar to ‘Butternut’, but sweeter and have a firm, deep, orange colored flesh. The flowers are orange and wide. As most cucurbits, the plant is monoecious and both male and female flowers are fully fertile.

The Cmv-2 gene was transferred to other Cucurbita plants using embyo rescue and backcross techniques. Firstly, Seminole Pumpkin was inbred for five cycles by artificial self-pollination. The F5 generation was then crossed with a proprietary inbred line of the yellow straightneck type, thus performing an interspecific cross between C. pepo and C. moschata. Embryo rescue techniques were used to obtain a few viable plants from this cross and self-pollinated seed of these plants. The detailed procedures are described in Example 1 below.

Interspecific progenies were obtained and used in several backcross breeding programs, thus producing an array of populations segregating for resistance to CMV derived from C. moschata var. Seminole pumpkin. The resistance conferred by the Cmv-2 gene is stable and can be transferred to other Cucurbita plants, in particular to other Cucurbita plants susceptible to the virus.

Accordingly, in one embodiment, the Cmv-2 gene is obtainable from a plant of C. moschata var. Seminole pumpkin, and the present invention discloses a method for producing a Cucurbita plant resistant to CMV comprising obtaining a plant of C. moschata var. Seminole pumpkin; crossing the plant of C. moschata var. Seminole pumpkin plant with a Cucurbita plant susceptible to CMV; rescuing an embryo resulting from the cross; regenerating a plant from the embryo; and selecting a plant that is resistant to CMV. In one embodiment, the present invention also discloses a Curcurbita plant obtainable by the method, wherein said plant comprises a Cmv-2 gene.

In one embodiment, the resistance to CMV is tested and assessed as described in Example 2 below. However, the person skilled in the art would also be able to use other protocols or methods to determine resistance to the virus. For example, viral RNA and protein can also be measured, for example as described in Kaplan et al. (1997) Virology 230: 343-349 and Mayers et al. (2005) MPMI 18: 428-434. Leaf-press blotting can also be carried out as described for example in Kaplan et al. (1997) Virology 230: 343-349.

The Cmv-2 gene confers resistance to various strains of CMV from different geographical locations. For example, the Cmv-2 gene confers upon a Cucurbita plant resistance to the Florida strain of CMV (University of Florida, Gainsville, Fla.). The Cmv-2 gene also confers upon a Cucurbita plant resistance to the California pepper strain of CMV and to the CMV-TL strain, a European strain of CMV (INRA, Virology Department, Montfavet 84, France). Other strains of CMV can also be tested as described herein.

In one embodiment, a plant of the invention is homozygous for a Cmv-2 gene. In one embodiment, the Cmv-2 gene is dominant. In one embodiment, the resistance to CMV according to the instant invention is monogenic. In one embodiment, the resistance to CMV according to the instant invention is monogenic and dominant.

A representative Cucurbita line comprising the Cmv-2 gene according to the present invention, inbred line 90-3588, was deposited with NCIMB, Aberdeen AB21 9YA, Scotland, UK under accession number NCIMB 41369 on Jan. 11, 2006. Plants of line 90-3588 are homozygous for the Cmv-2 gene. In one embodiment, the Cmv-2 gene is obtainable from a plant of line 90-3588. In one embodiment, a plant of the instant invention is a plant of inbred line 90-3588, representative seeds of which been deposited under accession number NCIMB 41369 (see Example 3 below).

The Cmv-2 gene is located at the chromosomal location of the gene conferring resistance to CMV in C. moschata var. Seminole pumpkin or in line 90-3588. In one embodiment, a plant comprising a Cmv-2 gene is identified and defined by an allelic test. An allelic test is performed by making a cross between a plant of a “tester” line (i.e. “tester” plant) and the plant to be tested, and determining the segregation ratio of plants resistant to CMV to plants susceptible to CMV in the progeny of the cross. Typically, F1 progeny plants and F2 progeny plants resulting from self-pollination of the F1 progeny plants are assessed. Progeny plants resulting from backcrosses with the parents and plants obtained after self-pollination of backcross progeny plants may also be assessed. The segregation ratio of resistant to susceptible plants can also be determined in subsequent generations of self-pollination and backcross with the parents. A tester plant is a plant comprising a Cmv-2 gene. In one embodiment, a tester plant is a plant of inbred line 90-3588, deposited with NCIMB under accession number NCIMB 41369.

In one embodiment, if a tester plant homozygous for the Cmv-2 gene and a plant to be tested homozygous for a single gene conferring resistance to CMV are used as parents in a cross, and only resistant plants are recovered in the F1 and F2 progenies (i.e. no segregation of resistant and susceptible plants), the plant to be tested comprises a Cmv-2 gene, and is defined as plant of the instant invention. Accordingly, in one embodiment, a plant of the present invention is a Cucurbita plant comprising a Cmv-2 gene, wherein when said Cucurbita plant is crossed with a plant of inbred line 90-3588 100% of F1 plants resulting from the cross are resistant to CMV, and 100% of the F1 plants produce 100% of F2 progeny plants resistant to CMV, when said Cucurbita plant is homozygous for a Cmv-2 gene (see Table 1). In this situation, if F1 or F2 progeny plants are backcrossed with the parents and the progeny plants resulting from the backcrosses are self-pollinated, only resistant plants are recovered in the progeny plants resulting from the backcrosses and from subsequent self-pollination.

In one embodiment, if a tester plant homozygous for the Cmv-2 gene and a plant to be tested heterozygous for the single gene conferring resistance to CMV are used as parents in a cross, and only resistant plants are recovered in the F1 progeny, and 100% of resistant F2 plants are recovered in the progeny of 50% of the F1 plants and a 3:1 ratio of resistant to susceptible F2 plants are recovered in the progeny plants of 50% of the F1 plants, the plant to be tested comprises a Cmv-2 gene, and is defined as plant of the instant invention. Accordingly, in one embodiment, a plant of the present invention is a Cucurbita plant comprising a Cmv-2 gene, wherein when said Cucurbita plant is crossed with a plant of inbred line 90-3588 100% of F1 plants resulting from said cross are resistant to CMV, and 50% of the F1 plants produce 100% of F2 progeny plants resistant to CMV and 50% the F1 plants produce a 3:1 ratio of resistant to susceptible F2 plants, when said Cucurbita plant is heterozygous for a Cmv-2 gene (see Table 1).

By contrast, if a tester plant homozygous for the Cmv-2 gene and a plant to be tested homozygous for a single gene conferring resistance to CMV are used as parents in a cross, and susceptible plants are found among F2 progeny plants, the plant to be tested contains a gene located at a different locus from that of the Cmv-2 gene. The plant to be tested thus does not comprise a Cmv-2 gene and is not a plant according to the instant invention. Susceptible plants are also found among F2 progeny plants if the plant to be tested is heterozygous for a single gene conferring resistance to CMV located at a different locus from that of the Cmv-2 gene. In this situation, if F1 or F2 progeny plants are backcrossed with the parents and the progeny plants resulting from the backcross are self-pollinated, susceptible plants are found in the progeny obtained after the self-pollination following the backcross.

The person skilled in the art would know how to carry out an allelic test, determine the appropriate number of progeny plants to be tested in each generation, and analyze the results of the test. For example, if the tester plant and the plant to be tested are both homozygous for a dominant single gene conferring resistance to the virus but located at different, unlinked chromosomal loci, one would expect only resistant plants in the F1 progeny, but a 15:1 segregation of resistant to susceptible plants in F2 progeny plants. In this case, if the tester plant is homozygous for the Cmv-2 gene and the plant to be tested is heterozygous for the single dominant gene located at a different, unlinked chromosomal locus one would expect only resistant plants in the F1 progeny, and a 3:1 segregation of resistant to susceptible plants in F2 plants of 50% of the F1 plants and a 15:1 segregation of resistant to susceptible plants in F2 plants of 50% of the F1 plants (see Table 1).

These segregation ratios may deviate from the above figures if the single dominant gene located at a different, unlinked chromosomal locus confers a milder or less stable resistance, for example moderate resistance to CMV as described herein. In this case, some of the plants comprising the single dominant gene located at a different, unlinked chromosomal locus may be rated as susceptible.

If the two chromosomal loci are linked, a higher ratio of resistant to susceptible plants would be expected.

The person skilled in the art would know how to carry out an allelic test, determine the appropriate number of progeny plants to be tested in each generation, and know how to analyze the results of the test. It is also understood that in rare cases a plant comprising a gene conferring resistance to CMV may be rated as susceptible due to aberrant seed germination or abnormal plant development. Such plants are however not considered in the allelic tests and in determining the segregation ratios and are generally eliminated in the disease testing.

Other types of crosses and allelic test are also within the skills of a person of the art and may also be carried out. Additional generations may also be evaluated. A tester cross can also be carried out with a tester plant heterozygous for a Cmv-2 gene and a plant to be tested heterozygous for a gene to be tested. The person skilled in the art would know how to carry out and interpret the result of these crosses.

TABLE 1
Expected results of allelic tests using a plant of inbred line 90-3588 as tester plant.

| Plant to be tested | | F1 progeny plants | F2 progeny plants |
|---|---|---|---|
| Plant comprising a Cmv-2 gene | Homozygous for gene | 100% R | 100% R |
| | Heterozygous for gene | 100% R | 100% R in 50% of the F1 progeny plants |
| | | | 3:1 ratio of R to S in 50% of the F1 progeny plants |
| Plant comprising a dominant single | Homozygous for gene | 100% R | 15:1 ratio of R to S |
| gene conferring resistance to CMV | Heterozygous for gene | 100% R | 15:1 ratio of R to S in 50% of the F1 progeny plants |
| located at a different, unlinked | | | 3:1 ratio of R to S in 50% of the F1 progeny plants |
| chromosomal location from that of a | | | |
| Cmv-2 gene | | | |

R: plant resistant to CMV, S: plant susceptible to CMV

In one embodiment, a plant of the invention is a Cucurbita plant comprising a Cmv-2 gene, wherein said plant is not a plant of C. moschata var. Seminole pumpkin. In one embodiment, a plant of the invention is not a C. moschata plant. In one embodiment, a plant of the invention is a plant of C. pepo, a plant of C. maxima or a plant of C. mixta. In one embodiment, a plant of the invention is a squash plant. In one embodiment a plant of the invention is a summer squash plant, a winter squash plant or a pumpkin plant. In one embodiment, a plant of the invention is a plant of C. pepo L.

Cucurbita pepo L. often refers to what is commonly known as the summer squash such as scallop, zucchini, straightneck and crookneck types, and winter squash such as acorn and pumpkin. Eight groups of edible C. pepo are known:

Pumpkin (C. pepo L. var. pepo L. Bailey) includes cultivars of creeping plants which produce spherical, oval or oblate fruit that is rounded or flat at the ends. The fruit of this group is grown to be eaten when ripe and sometimes is used as fodder.

Scallop (C. pepo L. var. clypeata Alefield) has a semi-shrubby habit, the fruit ranges from flat to almost discoidal, with undulations or equatorial margins, and it is eaten before maturity.

Acorn (C. pepo L. var. turbinata Paris) is both a shrubby and creeping plant with fruit which is obovoid or conical, pointed at the apex and longitudinally costate-grooved. The rind is soft, hence the fruit can be eaten in the ripe state.

Crookneck (C. pepo L. var. torticollia Alefield) is a shrubby type, with yellow, golden or white fruit which is claviform and curved at the distal or apical end and generally has a verrucose rind. It is eaten unripe since the rind and the flesh harden when ripe.

Straightneck (C. pepo L. var. recticollis Paris) is a shrubby plant with yellow or golden fruit and a verrucose rind similar to that of var torticollia.

Vegetable marrow (C. pepo L. var fastigata Paris) has creeper characteristics as a semi-shrub and has short cylindrical fruit that is slightly broader at the apex, with a smooth rind which hardens and thickens on ripening and which varies in colour from cream to dark green.

Cocozzelle (C. pepo L. var. Ionga Paris) has cylindrical, long fruit that is slender and slightly bulbous at the apex; it is eaten in the unripe state and one of the most common names is Cocozzelle.

Zucchini (C. pepo L. var. cylindrica Paris) is the most common group of cultivars at present. Like the previous group, the zucchini group has a strong affinity with the vegetable marrow and its origin is also recent (nineteenth century). Its plants are generally semi-shrubby and its cylindrical fruit does not broaden or else broadens only slightly. It is eaten as a vegetable in the unripe state.

In one embodiment, a plant of the instant invention is a plant of any one of the eight groups above, or any other groups from different classifications, wherein said plant comprises a Cmv-2 gene.

Squash plants usually develop a running vine on the soil but current summer squash cultivars have been developed in the form of a short compact bush, making them easier to grow in smaller spaces. On healthy winter squash plants, there is a canopy of large, reniform and serrated leaves, which may be without lobes or with very deep ones. Fruit flesh can be of various shade of yellow, or even from white to orange. The fruits may have a soft or a hard shell with colors from dull to bright. Summer squash show a great variety of shape, cylindrical, long, flat, etc., with sizes from small to large and colors from uniform to variegated. The flesh can range from white to yellow and, contrary to the winter squash that has a flesh finely grained, bear coarse grains.

Squash is a simple diploid species with twenty pairs of highly differentiated chromosomes. The plants are monoecious, with separate female and male flowers on the same plant. Usually, the first four or five flowers produced are male, then the next eight or so are female, followed by a few more male and female flowers. Male flowers have 3-5 erect stamens bunched within the corolla of 5 fused petals. Female flowers have 3 spreading stigma lobes and an immature fruit (external ovary) below the perianth. The spiny, sticky pollen requires insects for pollination. The primary pollinators are bees, particularly honeybees. Pollination generally occurs in the morning after the flowers open.

Based on the description of the present invention, the skilled person is able to recognize a Cucurbita plant of the instant invention, for example by performing an allelic test, for example an allelic test as described above. Accordingly, in one embodiment, the present invention also further discloses a method of identifying a Cucurbita plant of the instant invention comprising crossing a Cucurbita plant with a tester plant and determining the segregation ratio of the resistance to CMV in the resulting progeny. Based on the description of the present invention, the skilled person is able to recognize a plant resistant to CMV (also called herein “resistant plant”) and a plant susceptible to CMV (also called herein “susceptible” plant), for example using a disease testing as described herein.

In one embodiment, the Cmv-2 gene confers high level of resistance to CVM upon a Cucurbita plant, for example when tested and assayed as described in Example 2 below. Accordingly, in one embodiment, a plant resistant to CMV according to the present invention is a plant highly resistant to CMV (also called herein “highly resistant plant”). In one embodiment, the Cucurbita plant according to the present invention highly resistant to CMV is a plant of C. pepo. In one embodiment, said plant is a squash plant. In one embodiment, a highly resistant plant according to the present invention is identified and defined by an allelic test, for example an allelic test as described herein.

In a disease testing as described herein, a highly resistant plant shows insignificant symptoms of CMV infection. The first two true-leaves of a highly resistant plant may show symptoms of CMV infection, as in susceptible plants, but new leaves grow without any mosaic symptom, nor distortion and the plants do not show symptoms thereafter. Based on the description of the present invention, the skilled person is able to recognize a plant of the instant invention highly resistant to CMV, as example by using a disease testing as described herein. An example of a C. pepo plant highly resistant to CMV is a plant of inbred line 90-3588, representative seed of which being deposited under accession number NCIMB 41369, or a plant of hybrid RSQ2193 described herein. Accordingly, in one embodiment, a plant highly resistant to CMV shows the level of resistance as that of a plant of inbred line 90-3588 or of a plant of hybrid RSQ2193, for example in a disease test as disclosed herein.

In one embodiment, a C. pepo plant of the present invention is more resistant to CMV than currently available C. pepo plants, in particular when tested in a disease testing as described herein. For example, a C. pepo plant of the present invention is more resistant than a C. pepo plant moderately resistant to CMV (also called herein “moderately resistant plant”). In a disease testing described herein, a moderately resistant C. pepo plant shows reduced symptoms of CMV infection, such as symptoms of CMV infection beyond the first two true-leaves, sometimes on all leaves, delayed appearance of symptoms of CMV infection and symptoms remaining throughout plant growth. In some cases, moderately resistant plants may be overcome by the disease and may be rated as susceptible.

A C. pepo plant moderately resistant to CMV is for example a plant of squash hybrid ‘Dividend’ (a commercial variety of Syngenta Seeds, Inc.). Hybrid ‘Dividend’ comprises a Cmv-1 gene in the heterozygous state. ‘Dividend’ has also been described as tolerant to CMV. Accordingly, in one embodiment, a C. pepo plant of the present invention is more resistant to CMV than a C. pepo plant comprising a Cmv-1 gene. An allelic test between C. pepo plants comprising a Cmv-2 gene and C. pepo plants comprising a Cmv-1 gene was carried out and is described in Example 5 below. The allelic test indicates that the Cmv-2 gene is located at a different chromosomal location than the Cmv-1 gene.

Accordingly, in one embodiment, the Cmv-2 confers upon a C. pepo plant higher level of resistance to CMV than the Cmv-1 gene, in particular in a disease test as described herein. In one embodiment, the Cmv-2 confers upon a C. pepo plant a more stable level of resistance to CMV than the Cmv-1 gene, in particular in a disease test as described herein.

Disease tests were also carried out with C. moschata cv. Seminole Pumpkin and C. moschata cv. Nigerian Local. C. moschata cv. Nigerian Local is a winter squash, species C. moschata, described in Brown, 2003 (Euphytica 129: 253-258). The disease tests are described in Example 4 below. Plants of C. moschata cv. Seminole Pumpkin did not show any symptom of the disease and were rated as immune to CMV (also called herein “immune plant”). By contrast, plants of C. moschata cv. Nigerian Local showed symptoms of CMV infection on all plants tested and were rated as moderately resistant to CMV. The symptoms in C. moschata cv. Nigerian Local were limited to mosaic on all or most of the leaves; none of the tested plants was stunted (Table 9).

Accordingly, in one embodiment, a Cucurbita plant resistant to CMV can be further defined as highly resistant to CMV, moderately resistant to CMV or immune to CMV. A C. pepo plant susceptible to CMV is for example a plant of ‘Gentry’ (a commercial variety of Syngenta Seeds, Inc.).

The Cmv-2 gene was transferred to several types of Cucurbita plants. For example, the Cmv-2 gene was transferred to plants of the yellow crookneck type and to plants of the zucchini type. Based on the description of the present invention, the skilled person is also able to transfer a Cmv-2 gene to other Cucurbita plants lacking said gene, for example to a Cucurbita plant susceptible to CMV, and to produce a plant comprising the Cmv-2 gene. Accordingly, in one embodiment, the present invention further discloses a method of transferring a Cmv-2 gene to a Cucurbita plant lacking said gene comprising: a) obtaining a plant comprising a Cmv-2 gene; b) crossing it to a plant lacking said gene; c) obtaining plants of the cross of step b); d) selecting a plant of step c) comprising the Cmv-2 gene. In one embodiment, the method further comprises: e) back-crossing a plant resulting from step d) with a Cucurbita plant, and f) selecting for a Cucurbita plant comprising the Cmv-2 gene. In one embodiment, the method further comprises obtaining an inbred Cucurbita plant comprising the Cmv-2 gene, and, in one embodiment, the method further comprises crossing said inbred Cucurbita plant to another Cucurbita plant to produce a hybrid Cucurbita plant comprising the Cmv-2 gene. In one embodiment, the plant of step a) comprising a Cmv-2 gene is a plant of line 90-3588 or a progeny or ancestor of said plant. In one embodiment, the present invention discloses a Cucurbita plant obtainable by any one of the methods above, wherein the plant comprises the Cmv-2 gene. In one embodiment, such plant is a C. pepo plant, in one embodiment such plant is a C. pepo highly resistant to CMV. In one embodiment, the present invention also discloses methods of producing a Cucurbita plant comprising a Cmv-2 gene, for example comprising the steps set forth above.

In one embodiment, the present invention also discloses the use of a Cmv-2 gene to confer resistance to CMV upon a Cucurbita plant susceptible thereto. In one embodiment, the Cmv-2 gene is used in the homozygous state in said Cucurbita plant. In one embodiment, the present invention also discloses a method of conferring resistance to CMV to a Cucurbita plant susceptible thereto comprising: a) obtaining a plant comprising a Cmv-2 gene; b) crossing it to a plant susceptible to CMV; c) obtaining plants of the cross of step b); d) selecting a plant of step c) which is resistant to CMV. In one embodiment, the method further comprises: e) backcrossing a plant resulting from step d) with a Cucurbita plant, and f) selecting for a Cucurbita plant, which is resistant to CMV. In one embodiment, the method further comprises obtaining an inbred Cucurbita plant, which is resistant to CMV, and, in one embodiment, the method further comprises crossing said inbred Cucurbita plant to another Cucurbita plant to produce a hybrid Cucurbita plant, which is resistant to CMV. In one embodiment, the plant of step a) comprising a Cmv-2 gene is a plant of line 90-3588 or a progeny or ancestor of said plant. In one embodiment, the present invention discloses a Cucurbita plant obtainable by any one of the methods above, wherein the plant is resistant to CMV. In one embodiment, such plant is a C. pepo plant, in one embodiment such plant is a C. pepo highly resistant to CMV.

In one embodiment, a Cucurbita plant of the present invention comprises a Cmv-2 gene and a Cmv-1 gene. For example, such a plant is obtained by crossing a plant of inbred line 90-3588 and a plant of hybrid ‘Dividend’ and selecting for a plant comprising both genes, for example using an allelic test as described herein.

Commercial Cucurbita are generally F1 hybrids produced from the cross of two parental lines (inbreds). The development of hybrids requires, in general, the development of homozygous inbred lines, the crossing of these lines, and the evaluation of the crosses. Pedigree breeding and recurrent selection breeding methods are used to develop inbred lines from breeding populations. Breeders combine the genetic backgrounds from two or more inbred lines or various other germplasm sources into breeding pools from which new inbred lines are developed by selfing and selection of desired phenotypes. The new inbreds are crossed with other inbred lines and the hybrids from these crosses are evaluated to determine which of those have commercial potential. Plant breeding and hybrid development are expensive and time-consuming processes.

Accordingly, in one embodiment, the present invention also discloses a method of producing hybrid seed comprising crossing a plant of the present invention, wherein said plant is an inbred line, with a plant of another inbred line; and harvesting hybrid seed resulting from the cross. In one embodiment, both inbred lines in the cross are a plant of the present invention. In one embodiment, the present invention also discloses hybrid seed produced by the method above, wherein said hybrid seed comprises a Cmv-2 gene. In one embodiment, a hybrid seed of the present has inbred line 90-3588 as a parent. In one embodiment, a hybrid seed of the present invention is a seed of hybrid RSQ2193 (see Example 3 below).

Pedigree breeding starts with the crossing of two genotypes, each of which may have one or more desirable characteristics that is lacking in the other or which complements the other. If the two original parents do not provide all the desired characteristics, other sources can be included in the breeding population. In the pedigree method, superior plants are selfed and selected in successive generations. In the succeeding generations, the heterozygous condition gives way to homogeneous lines as a result of self-pollination and selection. Typically in the pedigree method of breeding five or more generations of self-pollination and selection is practiced: F1 to F2; F3 to F4; F4 to F5, etc.

A single cross hybrid results from the cross of two inbred lines, each of which has a genotype that complements the genotype of the other. The hybrid progeny of the first generation is designated F1. In the development of commercial hybrids only the F1 hybrid plants are sought. Preferred F1 hybrids can be more vigorous than their inbred parents. This hybrid vigor, or heterosis, can be manifested in many polygenic traits, including increased vegetative growth and increased yield.

Cucurbita plants can be easily cross-pollinated. A trait is readily transferred from one Cucurbita plant to another Cucurbita plant to further obtain commercial lines. The introgression of a trait into a line is for example achieved by recurrent selection breeding, for example by backcrossing. In this case, the line (recurrent parent) is first crossed to a donor inbred (the non-recurrent parent) that carries the trait. The progeny of this cross is then mated back to the recurrent parent followed by selection in the resultant progeny for the trait. After three, preferably four, more preferably five or more generations of backcrosses with the recurrent parent with selection for the trait, the progeny is heterozygous for the locus harboring the resistance, but is like the recurrent parent for most or almost all other genes (see, for example, Poehlman & Sleper (1995) Breeding Field Crops, 4th Ed., 172-175; Fehr (1987) Principles of Cultivar Development, Vol. 1: Theory and Technique, 360-376, incorporated herein by reference). Selection for the trait is carried out after each cross.

Accordingly, in one embodiment, a plant of the instant invention further comprises one or more traits, for example one or more transgenes. For example, in one embodiment, a plant of inbred line 90-3588 further comprises one or more traits, for example one or more transgenes. The traits are introgressed from another squash line, and the resulting plants have essentially all of the morphological and physiological characteristics of plants of inbred line 90-3588, in addition to the one or more traits introgressed into the inbred line. The invention also relates to seeds of an inbred line 90-3588 into which one or more traits have been introgressed and to plants of an inbred line 90-3588 into which one or more traits have been introgressed. The invention further relates to methods for producing a squash plant by crossing plants of an inbred line 90-3588 into which one or more traits have been introgressed with themselves or with another squash line. The invention also further relates to hybrid squash seeds and plants produced by crossing plants of an inbred line 90-3588 into which one or more traits have been introgressed with another squash line. Examples of traits transferred to inbred line 90-3588 include, but are not limited to, herbicide tolerance, resistance for bacterial, fungal, or viral disease, insect resistance.

In one embodiment, a Cucurbita plant of the present invention is male sterile. Male sterile lines do not produce viable pollen and cannot self-pollinate. By eliminating the pollen of one parental variety in a cross, a plant breeder is assured of obtaining hybrid seed of uniform quality. Therefore, the present invention discloses a male sterile Cucurbita plant, including seeds and materials of said plants and the progeny thereof. In one embodiment, a plant of the instant invention is a maintainer plant.

In one embodiment, a plant of the present invention is an inbred, a hybrid, or a dihaploid. In one embodiment, it is produced by pedigree breeding or by recurrent selection breeding. In one embodiment, a plant of the present invention has commercially acceptable agronomic characteristics.

In one embodiment, the present invention discloses a method of producing a fruit of a plant of the present invention comprising growing a plant of the present invention until a fruit is produced; and harvesting said fruit.

In one embodiment, the present invention discloses a method of producing seed of a plant of the present invention comprising: a) growing a plant of the present invention; b) allowing said plant to self-pollinate; c) harvesting seeds from said plant.

Cucurbita plants can also be propagated vegetatively using methods well-known in the art, for example in-vitro plant tissue culture, rooting side shoot or protoplast fusion. In one embodiment, a method of vegetatively propagating a plant of the present invention comprises: a) collecting tissue of a plant of the present invention; b) cultivating said tissue to obtain proliferated shoots; c) rooting said proliferated shoots to obtain rooted plantlets; d) growing plants from said rooted plantlets; and harvesting seeds from said plants.

Cucurbita plants of the present invention can also be transformed genetically with a gene of interest, using techniques well known in the art. Accordingly, the present invention also further disclose a Cucurbita plant according to the instant invention further such a gene of interest.

All references cited herein are incorporated by reference in the instant application in their entireties. The following examples are intended to provide illustrations of the application of the present invention. The following examples are not intended to completely define or otherwise limit the scope of the invention.

### EXAMPLES

### Example 1

### Interspecific Cross

A schematic description of the interspecific cross is provided below.

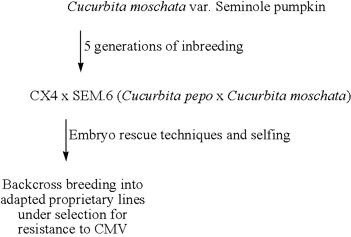

Detailed procedures are provided below:

Cultivation of Parental Materials and Artificial Crossing

Plants of both parents were grown in the greenhouse under standard cultural practices. CX4 is a proprietary inbred line of the yellow straightneck type. C. moschata var. Seminole pumpkin was inbred for five cycles by artificial self-pollination to obtain SEM.6. Plants were grown in polyethylene pots (Vol=10-15 L) filled with a soilless medium (peat moss, perlite, vermiculite, processed pine bark) and a balanced NPK (14:14:14), slow release fertilizer.

At flowering, female flowers of C. pepo were hand-pollinated with pollen from male flowers of C. moschata. The corolla of the male flower was removed with a twizer and the anthers were brushed on the stigma of the female flower for pollen transfer.

Seed Extraction and Embryo Explant

When the fruit set, 20 to 25 days after pollination, the fruit was harvested. The fruit was disinfected with ethanol and then cut in two halves along the vertical axis.

Seeds were extracted and dissected under a dissecting microscope. Embryos were separated from the seed using a forceps and a needle. The naked embryos were grown on V3 artificial medium (Tables 2-5) in a growth chamber under artificial conditions (t=25° C., 16 hours/day of light).

Seed dissection, embryo explant, and in vitro culture were performed under a laminar-flow sterile hood. All tools used for dissection and embryo transfer to the artificial medium were sterile.

Seedling Hardening

After roots form, the seedlings were transferred to polyethylene pots (4×4 cm) filled with peat. The seedlings were grown under artificial conditions in a growth chamber (t=25° C., 16 hours/day of light) and each pot was covered by a polyethylene bag, in order to maintain high humidity. The seedlings were watered daily as needed, with a water solution of the fungicide Previcur [2.5 mL/L]. A hole (3-5 mm) was punched in the polyethylene bag after five days, in order to decrease humidity and acclimate the seedlings prior to removal of the bag.

Plant Cultivation and Pollination

Once the roots had reached the borders of the pot, thus colonizing the medium, plants were transferred to the greenhouse or the open field and transplanted for self-pollination. Plants were grown under standard cultural practices and self-pollinated to create the first generation of C. pepo plants comprising the Cmv-2 gene.

TABLE 2
V3 medium used to culture the explanted embryos from
the interspecific cross C. pepo × C. moschata.

| Element | Volume | |
|---|---|---|
| Murashige & Skoog, macroelements [10×] | 100 | mL |
| Murashige & Skoog, microelements [100×] | 10 | mL |
| Morel vitamines | 2 | mL |
| Fe-EDTA | 10 | mL |
| Sucrose | 30 | g |
| Agar | 8 | g |
| H₂O | up to 1 | L |

| | |
|---|---|
| pH | 5.8 ± 0.1 |

TABLE 3
Preparation of 10× stock solution of Murashige & Skoog macroelements
to be used in the preparation of the V3 culture medium.

| | Quantity |
|---|---|
| Element | (g) |
| NH₄NO₃ | 16.5 |
| KNO₃ | 19.0 |
| CaCl₂2H₂O | 4.4 |
| MgSO₄7H₂O | 3.7 |
| KH₂PO₄ | 1.7 |
| H₂O | up to 1 L |

TABLE 4
Preparation of 100× stock solution of Murashige & Skoog microelements
to be used in the preparation of the V3 culture medium.

| | Quantity |
|---|---|
| Element | (mg) |
| H₃BO₃ | 620.0 |
| MnSO₄1H₂O | 1690.0 |
| ZnSO₄7H₂O | 860.0 |
| KI | 83.0 |
| Na₂MoO₄2H₂O | 25.0 |
| CuSO₄5H₂O | 2.5 |
| CoCl₂6H₂O | 2.5 |
| H₂O | up to 1 L |

TABLE 5
Preparation of 500× stock solution of Morel vitamins
to be used in the preparation of the V3 culture medium.

| | Quantity |
|---|---|
| Element | (mg) |
| Inositol | 5.0000 |
| Pyridoxine | 0.0500 |
| Nicotinic acid | 0.0500 |
| Thiamine | 0.0500 |
| Calcium panthotenate | 0.0500 |
| Biotin | 0.0005 |
| H₂O | up to 100 mL |

### Example 2

### Disease Testing

Inoculum Storage and Increase

The Florida strain of cucumber mosaic virus (CMV) was used in the disease testing. This strain of CMV is highly pathogenic to squash and is maintained in permanent storage at Syngenta, Naples, Fla. Fresh infected squash leaves were stored in polyethylene bags at t=−72° C. In order to increase the amount of inoculum available, susceptible squash plants were inoculated, as described below, and infected mature leaves harvested and stored, when severe symptoms appeared (mosaic and mottling of leaves, and stunting of plants).

Inoculation Method

Squash leaves from cold storage were ground in a tissue blender in cool 0.02 M phosphate buffer (Table 6) at pH 7.1±0.1. The inoculum solution was adjusted to a concentration of 1:6 (g of tissue:volume of buffer). The inoculum was maintained on ice for the duration of the inoculation process. Both cotyledons of plants at the first-true-leaf stage were inoculated by rubbing them with a gloved finger dipped into the inoculum solution. Gloves used were latex laboratory type and were changed every time the operator interrupted the inoculation procedure in order to perform other necessary steps (i.e.: moving flats).

Growing Conditions

Squash plants were sowed in 72-cell styrofoam speedling trays filled with a soilless medium (peat moss, perlite, vermiculite, processed pine bark). Trays were watered manually as needed and fertirrigated weekly with a soluble fertilizer (NPK 20:20:20). Inoculated trays were incubated in a growth chamber under artificial conditions (day t=85° C., night t=75° C., 16 hours of light/day). The growth chamber was organized with six shelves, three per layer, with upper and lower layers. Each shelf could accommodate six trays, side-by-side along their longer side. Each shelf had overhead fluorescent lights, adjustable in distance from the plants and lights were moved weekly during the growth cycle to maintain an average distance of 10 to 20 cm from the upper leaves of the plants, in order to prevent physiological stress to the plants and stretching and weakening of the seedlings.

Disease Assessment

Plants were rated for resistance to CMV two to three weeks after inoculation, once the susceptible checks show very severe mosaic and mottling of the leaves, and stunting of the plants. Single plants were counted by plot as resistant or susceptible.

Susceptible plants were identified when they showed the following symptoms of CMV infection at the seedling stage, depending on the severity of the infection:
Malformation and drastic reduction in size of leaves.
Development of a yellow mosaic pattern on leaves, followed by extensive necrosis under severe conditions.
Under severe conditions, stunting of susceptible plants.

Resistant plants were identified as follows:

A. Highly resistant plants showed insignificant symptoms of CMV infection:
The first two true-leaves may show symptoms of CMV infection, as in susceptible plants.
New leaves grow without any mosaic symptom, nor distortion. The plants do not show symptoms thereafter.

B. Moderately resistant plants showed reduced symptoms of CMV infection:
Symptoms of CMV infection beyond the first two true-leaves, sometimes on most or all leaves.
Delayed appearance of symptoms of CMV infection and symptoms remaining throughout plant growth.
In some cases, moderately resistant plants may be overcome by the disease and may be rated as susceptible.

C. Immune plants showed no symptoms of CMV infection:
None of the leaves nor any other organ of the plant may show symptoms of CMV infection, such as mosaic or plant stunting.

Variability in the experiment was reduced by eliminating at inoculation all seedling that germinated more than three days later than the majority and by applying fungicides for prevention of fungal diseases. Pathogens that might infect plants in the growth chamber in Florida include gummy stem blight, caused by Didymella bryoniae, and powdery mildew, caused by Sphaerotheca fuliginea.

Experimental Design

Cultigens (cultivars, experimental hybrids, and experimental inbreds) were planted in 30-plants plots (FIG. 1). Cultigens were randomly assigned to plots and plots (two per tray) were sequentially allocated to shelves in the growth chamber. Susceptible checks were planted in each tray with a frequency of 16.6% (1 plant every 6). Resistant checks were planted as 30-plants plots with a minimum frequency of one plot on each shelf of the growth chamber. When F2 and backcross segregating populations were screened, instead, single-plant plots were used.

TABLE 6
Preparation of 10× stock solution of 0.02 M phosphate
buffer to be used in inoculating CMV on squash seedlings.

| Element | Quantity (g) |
|---|---|
| KH₂PO₄ | 3.4 |
| K₂PO₄ | 8.7 |
| H₂O | up to 750 mL |
| pH | 7.1 ± 0.1 |

[image]

### Example 3

### Hybrid RS02193

Hybrid RS02193

RSQ2193 is an experimental hybrid of summer squash (Cucurbita pepo) of yellow crookneck type. RSQ2193 is adapted to the Southeastern United States, where it produces high yields of high quality fruit (Table 7). RSQ2193 is similar in fruit type to ‘Gentry’ (a commercial variety of Syngenta Seeds, Inc.) but has better fruit quality, resistance to a wider spectrum of diseases, and a more vigorous plant. The parents of RSQ2193 are inbred lines 90-3588 and 90-3590.

Plants of RSQ2193 are open and upright to semi-prostrate. These plants may produce a few lateral branches. Leaves and stems are medium green and the peduncles have fewer and smaller spines than comparable cultivars such as ‘Sunglo’ (a commercial variety of Syngenta Seeds, Inc.) and ‘Gentry’. The leaves are lobed, with denticulate flat margins and rounded apex. RSQ2193 carries orange male and female flowers, both fertile.

Fruit of RSQ2193 are yellow with a green peduncle and a semi-crooked neck. The fruit skin is very smooth, almost glabrous, with a very glossy finish. The blossom-end scar is small and flat to slightly protruded. The fruit are clubby, with a neck approximately ⅔ thinner than the main bulb of the fruit. The bulb is fairly elongated. The flesh quality at the marketable stage is comparable to ‘Gentry’, thus having a fine, dry, and soft texture.

Seeds of RSQ2193 are cream in color with a slightly darker rim around the edges. Seeds measure approximately 14 mm in length, 8 mm in width, and 3 mm in thickness. Seed coat is smooth, dull, and has curved and rounded margins. Seeds separate easily from the pulp at seed harvest.

RSQ2193 is highly resistant to cucumber mosaic virus (CMV) (Table 8), and powdery mildew caused by Sphaerotheca fuliginea. The resistance to CMV present in RSQ2193 is from C. moschata var. Seminole pumpkin, and has been transferred to this hybrid through interspecific cross as described in the instant application. RSQ2193 is homozygous for the Cmv-2 gene.

TABLE 7
Yield data collected in grower's fields in Georgia
in the Summer of 2005 for RSQ2193. Destiny III and Prelude
II are commercial varieties of Seminis, Inc.

| | | Yield (Boxes/acre) | | |
|---|---|---|---|---|
| Location | Cultivar | No. 1^{a} | No. 2^{a} | Total |
| 1 | RSQ2193 | 456 | 572 | 1028 |
| 1 | Destiny III | 391 | 526 | 917 |
| 2 | RSQ2193 | 334 | 467 | 801 |
| 2 | Gentry | 362 | 517 | 879 |
| 2 | Prelude II | 301 | 482 | 783 |
| 3 | RSQ2193 | 371 | 457 | 828 |
| 3 | Gentry | 411 | 476 | 887 |
| 3 | Prelude II | 380 | 443 | 823 |
| 4 | RSQ2193 | 385 | 448 | 833 |
| 4 | Gentry | 393 | 521 | 914 |
| 4 | Prelude II | 349 | 466 | 815 |
| 4 | Destiny III | 362 | 393 | 755 |
| 5 | RSQ2193 | 483 | 581 | 1064 |
| 5 | Prelude II | 467 | 564 | 1031 |

^{a}No. 1, #2 = Fruit quality categories, as determined by the ‘USDA-ARS Standards for Grades of Summer Squash’ (Effective Jan. 6, 1984 (Reprinted - January 1997)

Inbred Line 90-3588

Inbred line 90-3588 is a proprietary inbred line of summer squash (C. pepo) of yellow crookneck type. Line 90-3588 is a parent of hybrid RSQ2193 described above. Line 90-3588 is similar in fruit type to ‘Gentry’ and comprises in homozygous condition the Cmv-2 gene for resistance to cucumber mosaic virus.

Leaves of 90-3588 are entire, rounded, and medium green. Fruit of line 90-3588 are yellow with a green peduncle and a crooked neck. Seeds of line 90-3588 are cream in color with a slightly darker rim around the edges. Seeds measure approximately 13 mm in length, 8 mm in width, and 3 mm in thickness. Seed coat is smooth, dull, and has curved and rounded margins. Seeds separate easily from the pulp at seed harvest.

The resistance to CMV present in the genetics of line 90-3588 is from C. moschata var. Seminole pumpkin, and has been transferred to this inbred line through interspecific cross as described in the instant application.

TABLE 8
Resistance of RSQ2193 and parental inbreds to CMV in
the screening performed in 2005 in Naples, Florida.

| No. Resistant/No. Inoculated | | | | | |
|---|---|---|---|---|---|
| Rep 1 | Rep 2 | Rep 3 | Rep 4 | Rep 5 | Rep 6 |

RSQ2193

| | | | | | | |
|---|---|---|---|---|---|---|
| Female Parent | 28/28 | 27/27 | 28/28 | 27/27 | 30/30 | 29/29 |
| Male Parent | 24/24 | 25/25 | 21/21 | 25/25 | 26/26 | 22/22 |
| Hybrid | 29/29 | 30/30 | 29/29 | 29/29 | 28/28 | 30/30 |

Checks

| | | | | | | |
|---|---|---|---|---|---|---|
| Resistant^{a} | 36/36 | 36/36 | 36/36 | 36/36 | 36/36 | 36/36 |
| Susceptible | 0/26 | 0/30 | 0/26 | 0/29 | 0/29 | 0/29 |

^{a}C. moschata var. Seminole pumpkin, original source of resistance used to introgress resistance into RSQ2193

### Example 4

### Disease Testing of C. moschata cv. Seminole Pumpkin and Nigerian Local

C. moschata cv. Seminole Pumpkin and C. moschata cv. Nigerian Local were tested for resistance to CMV as described in Example 2 above. Nigerian Local is a winter squash, species Cucurbita moschata, described in Brown, 2003 (Euphytica 129: 253-258).

Plants of C. moschata cv. Seminole Pumpkin were rated as immune to CMV and did not show any symptom of the disease. Plants of C. moschata cv. Nigerian Local were rated as moderately resistant to CMV, but showed symptoms of CMV infection on all plants tested. The symptoms in C. moschata cv. Nigerian Local were limited to mosaic on the leaves and none of the tested plants was stunted (Table 9).

TABLE 9
Level of resistance to CMV in C. moschata cv. Seminole Pumpkin
and Nigerian Local in a screening in Naples, Florida. Six
repetitions were carried out and plants were rated for resistance
to CMV. The number of plants rated in each of the repetitions is
indicated. The susceptible check used was an inbred line
susceptible to all diseases, internally named “Universal
susceptible” 90-3871.

| | | | | | |
|---|---|---|---|---|---|
| Rep 1 | Rep 2 | Rep 3 | Rep 4 | Rep 5 | Rep 6 |

C. moschata cv. Nigerian Local

| | | | | | | |
|---|---|---|---|---|---|---|
| Immune | 0 | 0 | 0 | 0 | 0 | 0 |
| Moderately resistant | 24 | 30 | 30 | 26 | 29 | 30 |
| Susceptible | 0 | 0 | 0 | 0 | 0 | 0 |
| Total tested | 24 | 30 | 30 | 26 | 29 | 30 |

C. moschata cv. Seminole Pumpkin

| | | | | | | |
|---|---|---|---|---|---|---|
| Immune | 29 | 30 | 30 | 30 | 29 | 24 |
| Moderately resistant | 0 | 0 | 0 | 0 | 0 | 0 |
| Susceptible | 0 | 0 | 0 | 0 | 0 | 0 |
| Total tested | 29 | 30 | 30 | 30 | 29 | 24 |

Susceptible Check

| | | | | | | |
|---|---|---|---|---|---|---|
| Immune | 0 | 0 | 0 | 0 | 0 | 0 |
| Moderately resistant | 0 | 0 | 0 | 0 | 0 | 0 |
| Susceptible | 29 | 30 | 30 | 30 | 29 | 24 |
| Total tested | 29 | 30 | 30 | 30 | 29 | 24 |

### Example 5

### Allelic Test

An allelic test was carried out between plants of a breeding line of C. pepo comprising a Cmv-2 gene and plants of a breeding line of C. pepo comprising a Cmv-1 gene.

In a disease testing as described in Example 2 above, the disease was sometimes capable of overcoming the resistance conferred by the Cmv-1 gene leading to difficulties in rating plants comprising a Cmv-1 gene. Such plants may thus be rated resistant or susceptible. By contrast, the Cmv-2 gene confers a higher and more stable level of resistance, leading to a clear rating as resistant. In addition, the phenotypic manifestation of symptoms on resistant plants was different. Individuals plants containing a Cmv-1 gene but not a Cmv-2 gene may have a resistant reaction on the first two leaves to develop, following inoculation, while the subsequent leaves may look susceptible and the entire plant may eventually stunt and collapse at a later time. Plants homozygous or heterozygous for a Cmv-2 gene, instead, may show initial symptoms on the inoculated leaves. Plants would recover and from the third true-leaf on they would not show any more symptom of CMV infection.

Based on this differential expression of resistance, and the stronger effect of the Cmv-2 gene, the expected phenotypic frequencies (Table 10) may not be easily observed in artificial tests for resistance. The higher level of resistance conferred by the Cmv-2 gene was indicated by the phenotypic rating of the F1 hybrid heterozygous at both loci (Table 11, Entry 5) and the inbred line homozygous dominant for the Cmv-2 gene and homozygous recessive at the Cmv-1 locus (Table 1, Entry 2).

In the F2 populations derived from a cross of an inbred line homozygous dominant for a Cmv-1 gene, and an inbred line homozygous dominant for a Cmv-2 gene, plants susceptible to CMV were recovered (Table 11). This demonstrates that Cmv-1 and Cmv-2 in the populations of C. pepo used are two different genes.

The segregation ratio recorded of 206:48 (resistant:susceptible) plants deviated the expected 15:1 segregation ratio for two single dominant genes. However, the F1 hybrid and the inbred lines carrying a Cmv-1 gene (in heterozygous and homozygous condition, respectively) also contained individuals that were rated as susceptible in the test.

In conclusion, the allelic test indicates that the Cmv-2 gene is located at a different chromosomal location than the Cmv-1 gene. The Cmv-2 gene also confers higher level of resistance to CMV upon a C. pepo plant than the Cmv-1 gene. The unbalanced segregation ratio recorded in the F2 population tested can be explained by the mild and unstable resistance conferred by the Cmv-1 gene, in absence of the Cmv-2 gene. Individuals that were homozygous recessive at the Cmv-2 locus would have received only mild and unstable resistance from the Cmv-1 gene, thus some times being rated as susceptible.

The population should have included 32 individuals heterozygous for the Cmv-1 gene and homozygous recessive for the Cmv-2 gene, 16 individuals homozygous dominant for the Cmv-1 gene and homozygous recessive for the Cmv-2 gene, and 16 individuals double recessive (phenotypic expectations base on Mendel's inheritance rules and equal effects of two unlinked genes). Because of the instability or mild expression of resistance due to the Cmv-1 gene, the phenotype of the double recessive individuals may have been confounded with that of several individuals pertaining to the other two genotypic classes. Furthermore, this hypothesis is strongly supported by the evidence that the inbred homozygous dominant for the Cmv-1 gene, and not carrying the Cmv-2 gene, showed susceptible individuals at a high frequency (0.42).

TABLE 10
Segregation of the Cmv-1 and Cmv-2 genes in Cucurbita pepo,
based on a two-gene independent inheritance model (Mendel).

| | |
|---|---|
| Genotype^{a} | Phenotype^{b} |

| | Cmv-1 | Cmv-2 | Freq^{c} | Expected^{c} | Possible^{e} |
|---|---|---|---|---|---|
| 1 | R | R | 1/16 | R | R |
| 2 | R | H | 2/16 | R | R |
| 3 | R | S | 1/16 | R | R/S |
| 4 | H | R | 2/16 | R | R |
| 5 | H | H | 4/16 | R | R |
| 6 | H | S | 2/16 | R | R/S |
| 7 | S | R | 1/16 | R | R |
| 8 | S | H | 2/16 | R | R |
| 9 | S | S | 1/16 | S | S |

^{a}Genotype at the Cmv-1 and Cmv-2 loci. R = homozygous dominant; S = homozygous recessive; H = heterozygous; Seg = segregating according to Mendel's laws.
^{b}Phenotypic data for resistance to CMV artificial inoculation. R = resistant; S = susceptible.
^{c}Expected genotypic frequency.
d Expected phenotype, based on Mendel's inheritance rules.
^{e}Possible phenotype, due to the mild and unstable resistance conferred by the Cmv-1 gene.

TABLE 11
Segregation of the Cmv-1 and Cmv-2 genes in Cucurbita pepo,
as measured in Naples, Florida.

| | | Genotype^{a} | | | Phenotype^{b} | | |
|---|---|---|---|---|---|---|---|
| | Gen^{c} | Cmv-1 | Cmv-2 | Tot^{d} | Res | Sus | Dead |
| Entry 1 | IB | R | S | 21 | 12 | 8 | 1 |
| Entry 2 | IB | S | R | 119 | 118 | 1 | 0 |
| Entry 3 | F1 | H | S | 29 | 21 | 6 | 2 |
| Entry 4 | F1 | S | H | — | — | — | — |
| Entry 5 | F1 | H | H | 8 | 8 | 0 | 0 |
| Entry 6 | F2 | Seg | Seg | 255 | 206 | 48 | 1 |

^{a}Genotype at the Cmv-1 and Cmv-2 loci. R = homozygous dominant; S = homozygous recessive; H = heterozygous; Seg = segregating according to Mendel's laws.
^{b}Phenotypic data for resistance to CMV artificial inoculation. Res = number of resistant plants; Sus = number of susceptible plants; Dead = number of dead plants.
^{c}Generation. IB = inbred; F1, F2 = F1 and F2 hybrids.
^{d}Total number of plants tested.

What is claimed is:

1. A Cucurbita plant comprising a Cmv-2 gene, wherein, when said Cucurbita plant is crossed with a plant of inbred line 90-3588, 100% of F1 plants resulting from said cross are resistant to CMV, and:
a) 100% of said F1 plants produce 100% of F2 progeny plants resistant to CMV, when said Cucurbita plant is homozygous for a Cmv-2 gene; or
b) 50% of said F1 plants produce 100% of F2 progeny plant resistant to CMV and 50% said F1 plants produce a 3:1 ratio of F2 progeny plants resistant to CMV to F2 progeny plants susceptible to CMV, when said Cucurbita plant is heterozygous for a Cmv-2 gene,
wherein said plant is not a plant of C. moschata cv. Seminole Pumpkin.

2. The Cucurbita plant of claim 1, wherein said plant is not a C. moschata plant.

3. The Cucurbita plant of claim 1, wherein said plant is a C. pepo plant, a C. maxima plant or a C. mixta plant.

4. The Cucurbita plant of claim 1, wherein said plant is a C. pepo L. plant.

5. The Cucurbita plant according to claim 1, wherein said Cmv-2 gene is obtainable from a plant of C. moschata cv. Seminole Pumpkin.

6. The Cucurbita plant according to claim 1, wherein said Cmv-2 gene is obtainable from a plant of line 90-3588, representative seed of which being deposited under accession number NCIMB 41369.

7. The Cucurbita plant of claim 1, wherein said plant is homozygous for said Cmv-2 gene.

8. The Cucurbita plant of claim 1, wherein said plant is an inbred line, a hybrid or a dihaploid.

9. The Cucurbita plant of claim 1, wherein said plant is male-sterile.

10. A Cucurbita plant of inbred line 90-3588, representative seed of which been deposited under accession NCIMB 41369.

11. Seed of a Cucurbita plant according to claim 1.

12. Seed of a Cucurbita plant according to claim 10.

13. Part of a Cucurbita plant according to claim 1.

14. Part according to claim 13, wherein said part is a cell, pollen, and ovule.

15. Part according to claim 13, wherein said part is a fruit.

16. Part of a plant according to claim 10.

17. A method of producing a fruit of a Cucurbita plant comprising:
a) growing a Cucurbita plant according to claim 1 until a fruit is produced; and
b) harvesting said fruit.

18. A method of producing seed of a Cucurbita plant comprising:
a) growing a Cucurbita plant according to claim 1;
b) allowing said Cucurbita plant to self-pollinate; and
c) harvesting seeds from said Cucurbita plant.

19. A method of vegetatively propagating a Cucurbita plant comprising:
a) collecting a tissue of a Cucurbita plant according to claim 1;
b) cultivating said tissue to obtain proliferated shoots;
c) rooting said proliferated shoots to obtain rooted plantlets.

20. A method for producing a Cucurbita plant comprising a Cmv-2 gene comprising:
a) crossing a Cucurbita plant according to claim 1 and a Cucurbita plant lacking a Cmv-2 gene;
b) obtaining a progeny plant from the cross of step a); and
c) selecting in said progeny of step b) a Cucurbita plant comprising a Cmv-2 gene.

21. A method of transferring a Cmv-2 gene to a Cucurbita plant lacking said gene comprising:
a) crossing a Cucurbita plant according to claim 1 and a Cucurbita plant lacking a Cmv-2 gene;
b) obtaining a progeny plant from the cross of step a); and
c) selecting in said progeny of step b) a Cucurbita plant comprising a Cmv-2 gene.

22. A method of conferring resistance to CMV upon a Cucurbita plant comprising:
a) crossing a Cucurbita plant according to claim 1 and a Cucurbita plant susceptible to CMV;
b) obtaining a progeny plant from the cross of step a); and
c) selecting in said progeny of step b) a Cucurbita plant resistant to CMV.

23. A method for producing hybrid seed comprising:
a) crossing a plant according to claim 1, wherein said plant is a plant of an inbred line, with a plant of another inbred line; and
b) harvesting hybrid seed resulting from the cross of step a).

24. Hybrid seed produced by the method of claim 23, wherein said hybrid seed comprises a Cmv-2 gene.

25. A method for producing hybrid seed comprising:
a) crossing a plant according to claim 10, with a plant of another inbred line; and
b) harvesting hybrid seed resulting from the cross of step a).

26. Hybrid seed produced by the method of claim 25.

27. Hybrid seed according to claim 25, wherein said hybrid seed is a seed of hybrid RSQ2193.

28. A method for producing a Cucurbita plant comprising a Cmv-2 gene comprising:
a) obtaining a plant of C. moschata cv. Seminole Pumpkin;
b) crossing said plant of C. moschata cv. Seminole Pumpkin plant with a Cucurbita plant lacking a Cmv-2 gene;
c) rescuing an embryo resulting from the cross of step b);
d) regenerating a plant from said embryo of step c); and
e) selecting a plant of step d) that is resistant to CMV;

29. A C. pepo plant highly resistant to CMV, wherein said C. pepo plant comprises a Cmv-2 gene.

30. Seed of a plant according to claim 29.

31. Part of a plant according to claim 29.
